SEQUENCE LISTING (7 sequences)

SEQ ID NO: 1 - DNA, 1516 bp, Artificial Sequence
  DNA sequence of a fusion gene prepared by ligating adhesin gene of H. pylori and A2 and B subunit genes of Vibrio cholerae toxin
atgaaaaaaacagctatcgcgattgcagtggcactggctggtttcgcttccgtggctcaagctgcatggaaaaaatgccttttaggcgcgagcgtggtggctttattagtgggatgcagcccgcatattattgaaaccaatgaagtcgctttgaaattgaattaccatccagctagcgagaaagttcaagcgttagatgaaaagattttgcttttaaggccagctttccaatatagcgataatatcgctaaagagtatgaaaacaaattcaagaatcaaaccgcgctcaaggttgaacagattttgcaaaatcaaggctataaggttattagcgtagatagcagcgataaagacgatttttcttttgcacaaaaaaaagaagggtatttggcggttgctatgaatggcgaaattgttttacgccccgatcctaaaaggaccatacagaaaaaatcagaacccgggttattattctccaccggtttggacaaaatggaaggggttttaatcccggctgggtttattaaggttaccatactagagcctatgagtggggaatctttggattcttttacgatggatttgagcgagttggacattcaagaaaaattcttaaaaaccacccattcaagccatagcggggggttagttagcactatggttaagggaacggataattctaatgacgcgatcaagagcgctttgaataagatttttgcaaatatcatgcaagaaatagacaaaaaactcactcaaaagaatttagaatcttatcaaaaagacgccaaagaattaaaaggcaaaagaaaccgagaattcgagccgtggattcatcatgcaccgccgggttgtgggaatgctccaagatcatcgatcagtaatacttgcgatgaaaaaacccaaagtctaggtgtaaaattccttgacgaataccaatctaaagttaaaagacaaatattttcaggctatcaatctgatattgatacacataatagaattaaggatgaattatgattaaattaaaatttggtgttttttttacagttttactatcttcagcatatgcacatggaacacctcaaaatattactgatttgtgtgcagaatcacacaacacacaaatatatacgctaaatgataagatattttcgtatacagaatctctagctggaaaaagagagatggctatcattacttttaagaatggtgcaatttttcaagtagaagtaccaagtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaagatataaaaagcccacctcagtgggcttttttgtggttcgatgatgagaagcaaccgttttgcccaaacatgtattactgcaagtatgatgtttttattccacatccttagtgcgtattatgtgctgcagt

SEQ ID NO: 2 - protein, 337 aa, Artificial Sequence
  Amino acid sequence translated from a fusion gene prepared by ligating adhesin gene of H. pylori and A2 subunit gene of Vibrio cholerae toxin.
MKKTAIAIAVALAGFASVAQAAWKKCLLGASVVALLVGCSPHIIETNEVALKLNYHPASEKVQALDEKILLLRPAFQYSDNIAKEYENKFKNQTALKVEQILQNQGYKVISVDSSDKDDFSFAQKKEGYLAVAMNGEIVLRPDPKRTIQKKSEPGLLFSTGLDKMEGVLIPAGFIKVTILEPMSGESLDSFTMDLSELDIQEKFLKTTHSSHSGGLVSTMVKGTDNSNDAIKSALNKIFANIMQEIDKKLTQKNLESYQKDAKELKGKRNREFEPWIHHAPPGCGNAPRSSISNTCDEKTQSLGVKFLDEYQSKVKRQIFSGYQSDIDTHNRIKDEL

SEQ ID NO: 3 - DNA, 37 bp, Artificial Sequence
  Oligonucleotide
ccgtggctcaagctgaatggaaaaaatgccttttagg

SEQ ID NO: 4 - DNA, 30 bp, Artificial Sequence
  Oligonucleotide
agaattctcggtttcttttgccttttaatt

SEQ ID NO: 5 - DNA, 25 bp, Artificial Sequence
  Oligonucleotide
agaattcgagccgtggattcatcat

SEQ ID NO: 6 - DNA, 27 bp, Artificial Sequence
  Oligonucleotide
actgcagcacataatacgcactaagga

SEQ ID NO: 7 - protein, 124 aa, Vibrio cholerae
MIKLKFGVFFTVLLSSAYAHGTPQNITDLCAESHNTQIYTLNDKIFSYTESLAGKREMAIITFKNGAIFQVEVPSSQHIDSQKKAIERMKDTLRIAYLTEAKVEKLCVWNNKTPHAIAAISMAN

(19) United States
(12) Patent Application Publication
(10) Pub. No.: US 2002/0045218 A1
(43) Pub. Date: Apr. 18, 2002

(54) Recombinant microorganism expressing an antigenic protein, adhesin

(75) Inventors: Byung-O Kim, Kyunggi-Do (KR); Byoung-Kwang Lee, Kyunggi-Do (KR); Suk-Won Yoon, Kyunggi-Do (KR); Seung-Kook Park, Kyunggi-Do (KR); Young-Hyo Yu, Kyunggi-Do (KR)

Correspondence Address: DARBY & DARBY P.C., 805 Third Avenue, New York, NY, 10022, US

(73) Assignee: BIOPHARM INC.

(21) Appl. No.: 09/760,234

(22) Filed: Jan. 11, 2001

Related U.S. Application Data
(63) Continuation of application No. 09/051,315, filed on Apr. 3, 1998, now abandoned.
Application No. 09/051,315 is a 371 of international application No. PCT/KR97/00091, filed on May 21, 1997.

(51) Int. Cl. C12P 21/02; C12N 1/21; C12N 9/00; C07H 21/04

(52) U.S. Cl. 435/69.2; 435/183; 435/252.33; 536/23.7

(57) ABSTRACT

The present invention relates to a recombinant expression vector containing a fusion gene which is prepared by ligating adhesion gene of H. pylori and A2 and B subunit genes of Vibrio cholerae toxin, and a process for preparing a chimeric protein employing a recombinant microorganism transformed with the said expression vector. The recombinant DNA which is designed for convenient expression and gene manipulation, may express a chimeric protein having excellent immunogenicity to H. pylori, which is stable in stomach, and penetrate mucous membrane of stomach easily, finally to stimulate production of sIgA. Accordingly, the chimeric protein expressed from the recombinant DNA may be used as an active ingredient of the diagnostic kit for H. pylori infection and preventive or therapeutic vaccine for H. pylori-associated diseases, and may be used in the production of anti-H. pylori antibody.

### BACKGROUND OF THE INVENTION

1. Field of the Invention

The present invention relates to a recombinant DNA coding for an antigenic protein of Helicobacter pylori, adhesin and a recombinant microorganism transformed therewith, more specifically, to a recombinant expression vector containing a fusion gene which is prepared by ligating adhesin gene of H. pylori and A2 and B subunit genes of Vibrio cholerae toxin, and a process for preparing a chimeric protein employing a recombinant microorganism transformed with the said expression vector.

2. Description of the Prior Art

Although gastritis-associated diseases such as gastritis, gastric ulcer and duodenal ulcer are caused by various etiological factors, they are mainly caused by Helicobacter pylori (hereinafter, referred to as H. pylori) colonizing in the junctional region of epithelial cells of stomach mucous membrane. It has been reported that 90% or more of Asians and 60% or more of Europeans are infected with H. pylori though there are local differences. Also, it has been known that recurrence of gastritis, gastric ulcer or duodenal ulcer is caused by drug-resistant H. pylori, which may give rise to the occurrence of gastric cancer(see: Timothy, et al., ASM News, 61:21(1995)).

So far, a variety of chemical therapeutic agents such as antibiotics and anti-ulcer agents have been used, in order to treat the gastricis-associated diseases caused by H. pylori. However, these drugs have revealed some drawbacks as followings: limitation in penetrating the mucous membrane of stomach, emergence of drug-resistant microorganisms, occurrence of reinfection and untoward effects of the drugs. Under the circumstances, there are strong reasons for exploring and developing alternative drugs for the control of H. pylori employing new therapeutic approach, e.g., immunological therapy which can substitute for chemical therapy.

Recently, in order to solve the said problems, studies on the development of vaccines to H. pylori has been carried out. As a result, diagnostic agents of H. pylori infection and preventive vaccines for H. pylori-associated diseases have been developed, employing genes coding for antigenic determinants of H. pylori, e.g., urease gene(see: Timothy, et al., Infection and Immunity, 59:1264(1991)), flagella gene(see: Leying, et al., Molecular Microbiology, 6:2863(1992)), adhesin gene(see: Evans, et al., Journal of Bacteriology, 175:674(1993)), superoxide dismutase gene (see: Christiane, et al., Infection and Immunity, 61:5315(1993)), catalase gene and vacuolating cytotoxin gene(see: Timothy, et al., Infection and Immunity, 58:603(1990)), some of which have been tested in preclinical phase.

However, they have not been manufactured up to now, owing to the following disadvantages: a vaccine employing an urease gene has poor immunogenicity; a vaccine employing a vacuolating cytotoxin gene may have toxicity of cytotoxin itself, though it provides excellent immunogenicity; a vaccine employing a non-toxic varient gene of the vacuolating cytotoxin gene does not have effects on all over the strains of H. pylori, since the non-toxic varient gene does not appear in all H. pylori; and, a vaccine employing adhesin gene, despite its excellent immunogenicity, does not have good efficacy, since it does not stimulate production of secretory IgA(“sIgA”).

In general, H. pylori is controlled by sIgA not by serum IgG, since it colonizes in the junctional region of epithelial cells of stomach mucous membrane. However, since the aforesaid vaccines can not penetrate the mucous membrane of stomach easily, they are not able to stimulate mucosal immune system, which, in turn, results in decreased production of sIgA. Thus, serious problems have occurred that immunological effects of the vaccines against H. pylori decrease and the vaccines are easily denaturated by gastric acid(pH 1-2) to provide poor activities.

### SUMMARY OF THE INVENTION

Since vaccines employing H. pylori gene alone have the said various disadvantages, the present inventors have made an effort to prepare a chimeric protein ligated with a protein which can penetrate mucous membrane of stomach easily and stimulate mucosal immune system to produce sIgA, as a fusion partner, for the purpose of using the chimeric protein as a potential vaccine for H. pylori.

Thus, the present inventors, for the first time, prepared a chimeric protein expressed from a recombinant DNA which contains adhesin gene coding for an antigenic determinant of H. pylori and A2 and B subunit genes of Vibrio cholerae toxin as an adjuvant. Also, they discovered that the chimeric protein successfully solves the problems of the conventional vaccines and can be used as an effective vaccine for H. pylori, based on its excellent immunogenicity for H. pylori, stability under stomach environment, and penetrating property through stomach mucous membrane to stimulate sIgA production.

A primary object of the invention is, therefore, to provide a DNA sequence prepared by ligating adhesin gene of H. pylori and A2 and B subunit genes of Vibrio cholerae toxin, and an amino acid sequence translated therefrom.

The other object of the invention is to provide an expression vector comprising the said DNA sequence and a recombinant microorganism transformed therewith.

Another object of the invention is to provide a process for preparing a chimeric protein consisting of adhesin, an antigenic determinant of H. pylori and A2 and B subunits of Vibrio cholerae toxin from the said microorganism.

### BRIEF DESCRIPTION OF THE DRAWINGS

The above and the other objects and features of the present invention will become apparent from the following descriptions given in conjunction with the accompanying drawings, in which:

FIG. 1 shows a DNA sequence(SEQ ID NO:1) of a fusion gene prepared by ligating adhesin gene of H. pylori and A2 and B subunit genes of Vibrio cholerae toxin, and an amino acid sequence(SEQ ID NO:2) translated therefrom.

FIG. 2 is a schematic diagram showing construction strategy of an expression vector for adhesin of H. pylori, pHA022.

FIG. 3 is a photograph showing 1% agarose gel electrophoresis pattern of pTE105 plasmid, a fusion gene and pHAO22 expression vector, after DsaI/PstI digestion.

FIG. 4(A) is a photograph showing 15% SDS-PAGE pattern of whole cell lysate of E. coli transformed with pHA022 expression vector.

FIG. 4(B) is a photograph showing the result of Western blot analysis corresponding to SDS-PAGE of FIG. 4(A).

### DETAILED DESCRIPTION OF THE INVENTION

The present inventors gave an attention to the following characteristics of a toxin gene of Vibrio cholerae in the course of searching for a fusion partner of H. pylori gene: A gene of Vibrio cholerae toxin consists of genes coding for three subunits of A1, A2 and B. A1 subunit has a toxic activity of the toxin, and A2 and B subunits bind to host cell to stimulate production of sIgA and guarantee stability of the protein under the surrounding environment. Also, vaccines employing A2 and B subunit genes of cholera toxin can be applied to human body, due to its tolerable characteristics, while various vaccines employing intact cholera toxin gene as a fusion partner, owing to toxic property of A1 subunit, can not be used directly for human body. Further, studies on a vaccine employing A2 and B subunits of cholera toxin as a fusion partner, have revealed that production of sIgA and serum IgG is stimulated when a chimeric protein prepared by ligating A2 and B subunit genes of cholera toxin with adhesin gene of Streptococcus mutans is orally administrated(see: Hajishengallis, et al., The Journal of Immunology, 154:4322(1995)).

Grounded on the afore-mentioned knowledges, the present inventors prepared a chimeric protein employing adhesin gene of H. pylori whose product has an excellent immunogenicity, in order to stimulate production of antibody to H. pylori: First, adhesin gene of H. pylori and A2 and B subunit genes of Vibrio cholerae toxin were prepared by employing polymerase chain reaction(PCR) technique, respectively. Then, each gene was cleaved with EcoRI and said two genes were ligated with T₄ DNA ligase. The fusion gene thus prepared was cleaved with DsaI and PstI, and inserted into pTE105 plasmid(see: Korean patent No.102993, KCCM-10027) to prepare an expression vector, pHAO22. Then, E. coli was transformed with the expression vector, the recombinant E. coli was cultured, and a chimeric protein of adhesin of H. pylori and A2 and B subunits of Vibrio cholerae toxin was obtained.

In describing the present invention, the term ‘adhesin’ is employed to mean an antigenic determinant of H. pylori whose (“please further describe physicochemical property of adhesin”). Further, in describing the amino acid sequence of the present invention, the term ‘proteins consisting of functionally equivalent amino acids’ is employed to mean all proteins substituted by the combinations such as Gly, Ala; Val, Ile, Leu; Asp, Glu; Asn, Gln; Ser, Thr; Lys, Arg; and, Phe, Tyr among the amino acid sequences of the chimeric protein, and in describing the nucleotide sequence of the invention, the term ‘functional equivalents’ means all genes comprising nucleotide sequences coding for all the said combinations.

The chimeric protein thus prepared may be used as an active ingredient of the diagnostic kit for H. pylori infection and preventive or therapeutic vaccine for H. pylori-associated diseases, and used in the production of anti-H. pylori antibody.

The present invention is further illustrated in the following examples, which should not be taken to limit the scope of the invention.

### EXAMPLE 1

### Isolation of chromosomal DNA from H. pylori

H. pylori 11637 RPH 13487(ATCC 43504) was cultured in the BHI(brain heart infusion) liquid medium(consisting of 10 mg/ml vancomycin, 5 mg/ml trimetofrim and 4 mg/ml amphotericin B) containing 5% horse serum, and incubated for 72 hours under an environment of 10%(v/v) Co₂. Then, chromosomal DNA was isolated from the cultured cells by the conventional method in the art.

### EXAMPLE 2

### Synthesis, Purification and Analysis of Oligonucleotides

Two oligonucleotides of 37-mer and 30-mer as followings, were synthesized to amplify adhesin gene of H. pylori by PCR technique described in Example 3 below:

5′-CCGTGGCTCAAGCTGAATGGAAAAAATGCCTTTTAGG-3′ (SEQ ID NO:3)

5′-AGAATTCTCGGTTTCTTTTGCCTTTTAATT-3′ (SEQ ID NO:4)

In an analogous manner, two oligonucleotides of 25-mer and 27-mer as followings, were synthesized to amplify A2 and B subunit genes of Vibrio cholerae toxin:

5′-AGAATTCGAGCCGTGGATTCATCAT-3′ (SEQ ID NO:5)

5′-ACTGCAGCACATAATACGCACTAAGGA-3′ (SEQ ID NO:6)

In this connection, the oligonucleotides were synthesized employing an automatic nucleotide synthesizer (Pharmacia-LKB Biotechnology, Uppsala, Sweden).

The oligonucleotides thus prepared were reacted with TTD(thiophenol/triethylamine/dioxane=1/2/2, v/v/v) solution in a silica matrix, washed with methanol and ethanol sufficiently, and treated with strong ammonia water to separate the synthesized oligonucleotides from the silica matrix. To the oligonucleotide solutions thus obtained was further added strong ammonia water. Then, the solutions were left to stand at 50° C. for 12 hours, and concentrated under a reduced pressure with gas removal to reach a final volume of 0.5 ml. And then, using the oligonucleotides thus concentrated, primary purification was carried out with acetonitrile/triethylamine buffer employing a SEP-PAK cartridge(Waters Inc., USA), and electrophoresis was performed using 15% polyacrylamide gel(in TE-borate, pH 8.3). After electrophoresis, oligonucleotides were visualized under shortwave ultraviolet rays, and only the gel fragments corresponding to the oligonucleotides were cleaved. Then, oligonucleotides were electroeluted from the gel fragments, while remaining salts with acetonitrile/triethylamine buffer employing SEP-PAK cartridge connected with a syringe to purify each oligonucleotide. Oligonucleotides thus purified were labelled with γ-[³²p]-ATP employing T₄ polynucleotide kinase(New England Biolabs, #201S, USA) and the nucleotide sequences were determined by Maxam and Gilbert's nucleotide sequencing method(see: Maxam, A. M. & Gilbert, W., Proc. Natl. Acad. Sci., USA, 74:560-564(1977)).

### EXAMPLE 3

### Polymerase Chain Reaction(PCR)

To the solution containing template DNA(10 ng), 10 μl of 10×Taq polymerase buffer(10 mM Tris-HCl(pH 8.3) containing 500 mM KCl, 15 mM MgCl₂ and 0.1%(v/v) gelatin), 10 μl of dNTP's mixture(containing an equimolar concentration of 1.25 mM dGTP, DATP, dTTP and dCTP), 2 μg of each primer (oligonucleotides synthesized in Example 2) and 0.5 μl of Ampli Taq DNA polymerase(Perkin-Elmer Cetus, USA), was added distilled water to be a final volume of 100 μl. In order to prevent evaporation of the solution, 50 μl of mineral oil was added to the solution. In case of amplification of adhesin gene of H. pylori, chromosomal DNA of H. pylori isolated in Example 1 was used as a template DNA, and oligonucleotides synthesized in Example 2, i.e., 37-mer and 30-mer, were used as primers; and, in case of amplification of A2 and B subunit genes of Vibrio cholerae toxin, chromosomal DNA of Vibrio cholerae was used as a template DNA, and oligonucleotides synthesized in Example 2, i.e., 25-mer and 27-mer, were used as primers.

Denaturation(95° C., 1 minute), annealing(55° C., 1 minute), and extension(72° C., 2 minute) were carried out for 30 cycles in a serial manner, using Thermal Cycler TM(Cetus/Perkin-Elmer, USA), and final reaction was carried out at 72° C. for 10 minutes. And then, in order to remove polymerase, the equal volume of phenol/chloroform mixture(1:1(v/v)) was added to the reaction mixture, mixed well, and subsequently centrifuged. The supernatant thus obtained was transferred to a fresh tube. Then, 1/10 volume of 3M sodium acetate and 2 volume of 100% ethanol was added to the supernatant, mixed and centrifuged to obtain double-stranded nucleic acid. The nucleic acid was dissolved in 20 μl of TE buffer for later use.

### EXAMPLE 4

### Construction of an Expression Vector, pHA022

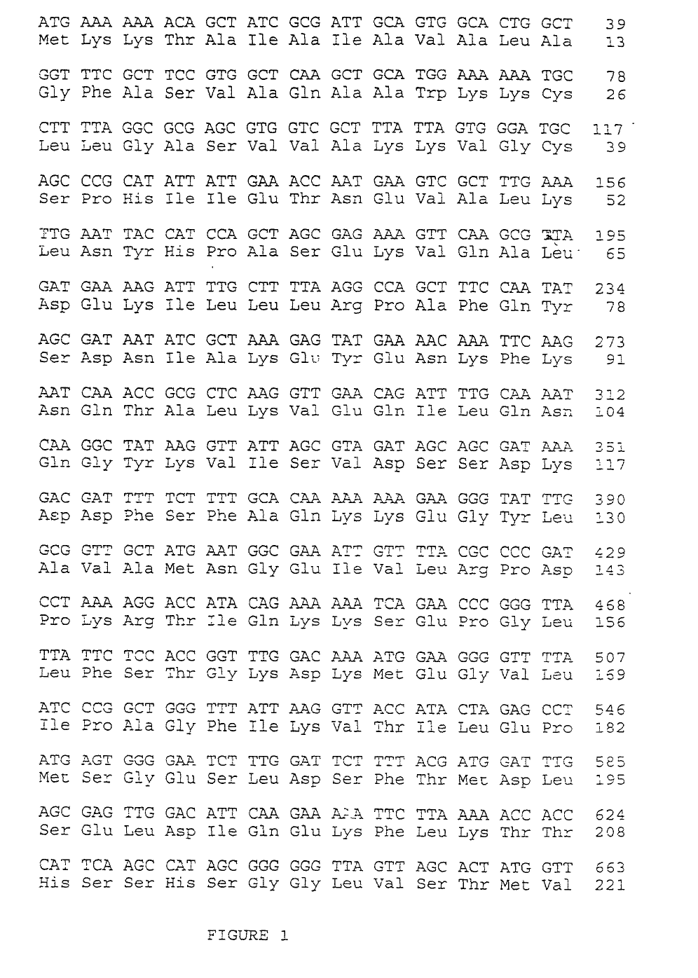

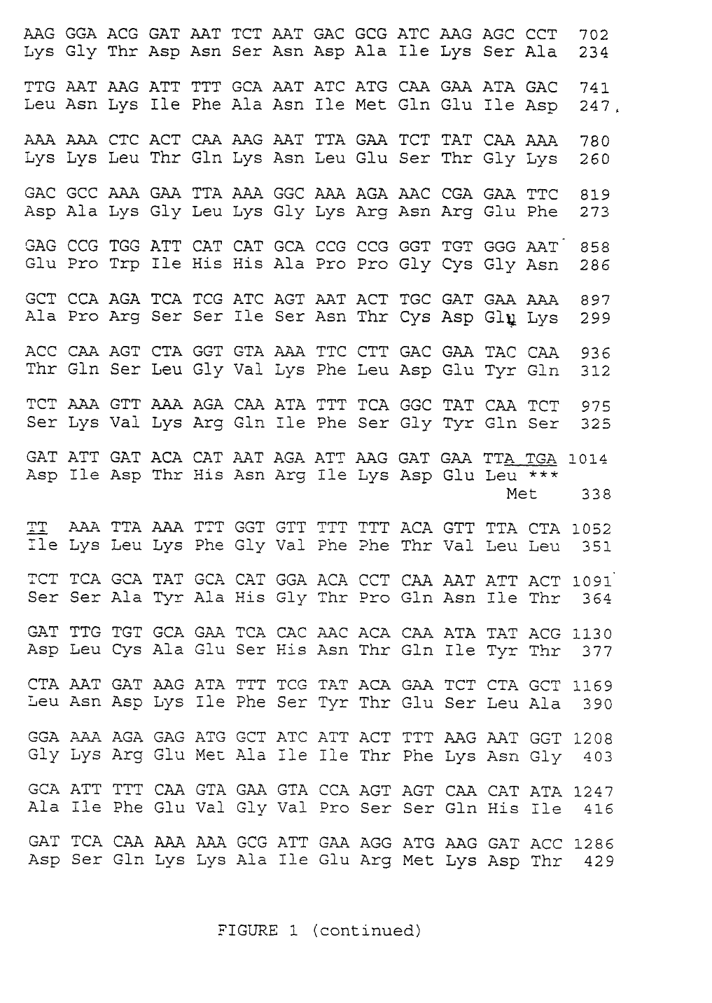

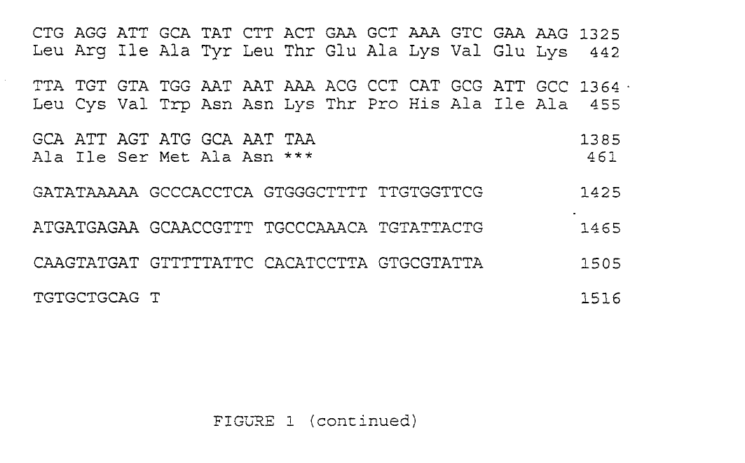

The adhesin gene of H. pylori and the A2 and B subunit genes of Vibrio cholerae toxin amplified in Example 3, respectively, were digested with EcoRI, respectively. Each of 1 μg of H. pylori DNA and Vibrio cholerae DNA was mixed. Then, 3 μl of 10× concentrated solution for fusion(600 mM Tris-HCl buffer(pH 7.5) containing 10 mM DTT and 100 mM MgCl₂), 1 μl of 10 mM ATP and 10 unit of T₄ DNA ligase were added to the DNA mixture to reach a final reaction volume of 30 μl, and held at 14° C. for 16 hours. After 1% agarose gel electrophoresis of the reaction product, a fusion gene of about 1.5 kb was obtained and its nucleotide sequence was determined(see: FIG. 1). In FIG. 1, nucleotide sequence of base position 1 to 54 corresponds to signal peptide sequence of the adhesin, and nucleotide sequence of base position 1010 to 1070 corresponds to signal peptide sequence of the B subunit of Vibrio cholerae toxin.

The fusion gene having the nucleotide sequence thus determined was double-digested with DsaI and PstI, and inserted into pTE105 plasmid vector(see: Korean patent No.102993, KCCM-10027) double-digested with the said restriction enzymes to prepare a circular plasmid which was designated as ‘pHA022’. FIG. 2 is a schematic diagram showing the construction strategy of pHA022.

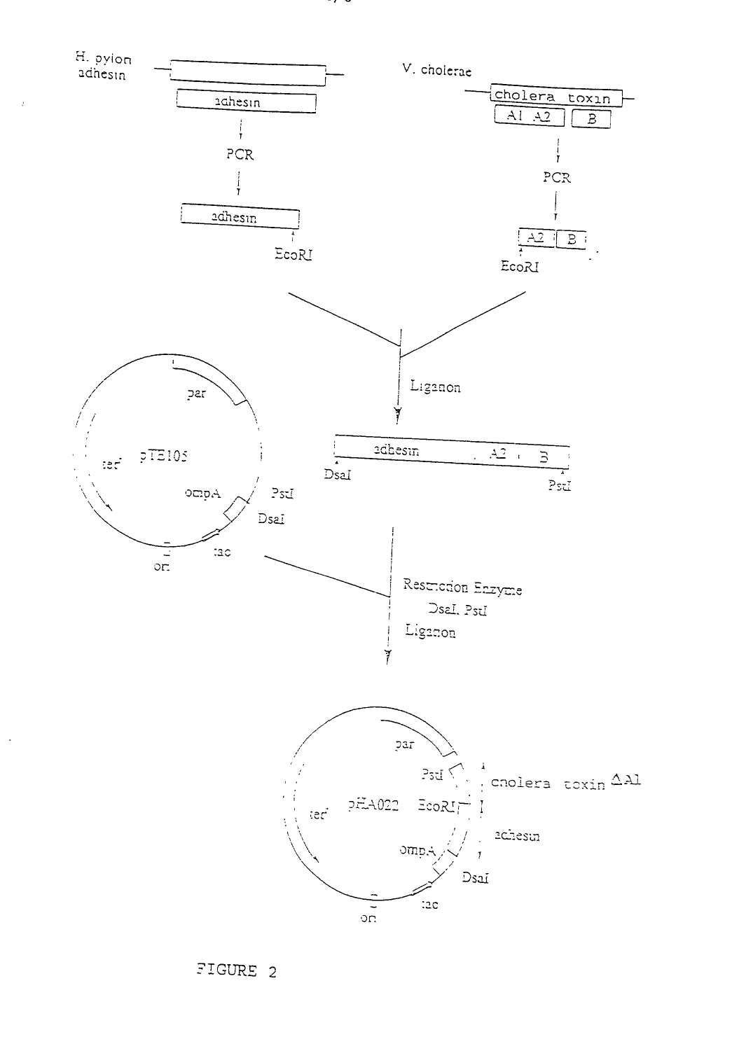

FIG. 3 is a photograph showing that the fusion gene was- correctly inserted into pTE105 plasmid. In FIG. 3, lane 1 shows 1kb ladder of DNA size marker; lane 2 shows pTE105 plasmid after DsaI/PstI digestion; lane 3 shows adhesin gene; lane 4 shows A1 subunit gene of cholera toxin; lane 5 shows a fusion gene digested with DsaI/PstI; and, lane 6 shows pHA022 digested with DsaI/PstI. As shown in FIG. 3, it was found that the fusion gene was correctly inserted into pTE105 plasmid. Further, 1% agarose gel electrophoresis revealed that the pHA022 expression vector has unique restriction site for each restriction enzyme.

### EXAMPLE 5

### Preparation of Transformant

In order to transform host cell with the pHA022 expression vector, E. coli JM101 was first inoculated in liquid LB medium, cultured at 37° C. until absorbance at 650 nm reached to a level of 0.25 to 0.5, and harvested, which was subsequently washed with 0.1M MgCl₂, and centrifuged. To the precipitate thus obtained were added solution containing 0.1M CaCl₂ and 0.05M MgCl₂, and the pHA022 expression vector prepared in Example 4, and incubated on ice. The cells were centrifuged again, and dispersed uniformly in the same solution(see: DNA Cloning Vol. I, A Practical Approach, IRL press, 1985). In this connection, all solutions and tubes were used after cooling at 0° C.

And then, 0.2 ml of the cell suspension thus obtained was added to petri dishes coated with liquid LB media containing 12.5 μg/ml of tetracycline, and cultured at 37° C. overnight to obtain transformant of E. coli JM101 harboring pHA022. The transformant thus prepared was designated as Escherichia coli DW/HP-222, and deposited with the Korean Culture Center of Microorganisms (KCCM), an international depository authority as deposition No. KCCM-10078 on Dec. 11, 1995.

### EXAMPLE 6

### Expression of a Chimeric Protein

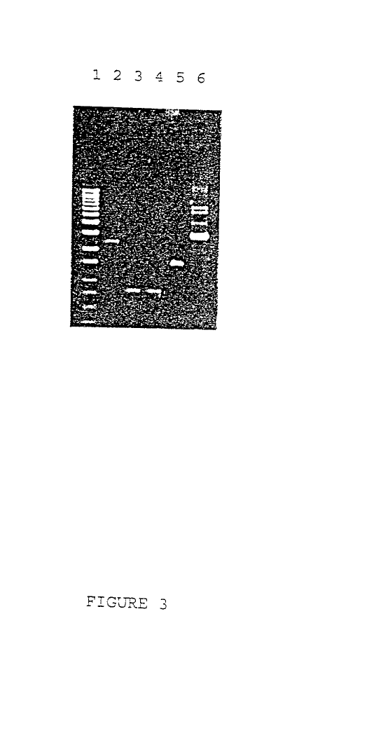

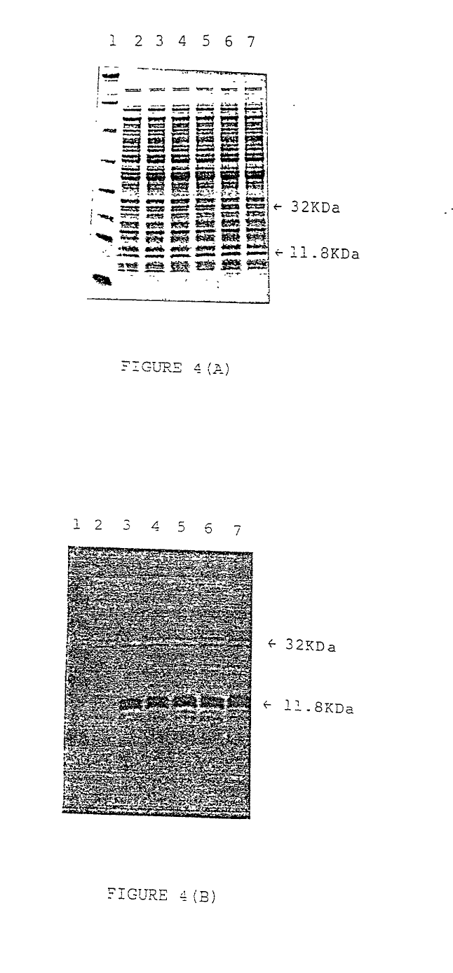

The transformed E. coli DW/HP-222 was cultured in liquid LB medium at 37° C. for 18 hours, centrifuged to harvest cells, resuspended in a buffer(10 mM Tris-HCl(pH 8.0) containing 0.16 Triton X-100, 2 mM EDTA and 1 mM PMSF), and lysed to prepare E. coli extract. Then, 15% SDS-PAGE was carried out with 10 μl of E. coli extract according to Laemmli's method(see: Laemmli, et al., Nature, 277:680(1970))(see: FIG. 4(A)).

In FIG. 4(A), lane 1 shows molecular weight standards such as 97 kDa, 66 kDa, 45 kDa, 31 kDa, 21.5 kDa, 14.4 kDa, and 6.5 kDa; lanes 2 to 7 show extract of DW/HP-222, E. coli harboring the pHA022 at 0, 1, 2, 3, 4 and 24 hours after IPTG(isopropyl β-D-thiogalactoside) induction, respectively; top arrow indicates locus of chimeric protein of adhesin of H. pylori and A2 subunit of a Vibrio cholerae toxin; and, bottom arrow indicates locus of B subunit of Vibrio cholerae toxin. FIG. 4(B) is a photograph showing the result of Western blot analysis corresponding to SDS-PAGE as shown in FIG. 4(A) with a 1:1000 dilution of polyclonal anti-cholera toxin serum.

As shown in FIGS. 4(A) and 4(B), it was found that the transformed E. coli DW/HP-222 successfully expresses a chimeric protein.

As clearly illustrated and demonstrated as aboves, the present invention provides an expression vector containing a recombinant DNA which is prepared by ligating adhesin gene of H. pylori and A2 and B subunit genes of Vibrio cholerae toxin, and a process for preparing a chimeric protein of adhesin of H. pylori and A2 and B subunits of Vibrio cholerae toxin, employing a recombinant microorganism transformed with the said expression vector. The recombinant DNA which is designed for convenient expression and gene manipulation, may express a chimeric protein having excellent immunogenicity to H. pylori, which is stable in stomach, and penetrate mucous membrane of stomach easily, finally to stimulate production of sIgA. Accordingly, the chimeric protein expressed from the recombinant DNA may be used as an active ingredient of the diagnostic kit for H. pylori infection and preventive or therapeutic vaccine for H. pylori-associated diseases, and may be used in the production of anti-H. pylori antibody.

What is claimed is:

1. A recombinant DNA comprising a fusion gene which is prepared by ligating adhesin gene of Helicobacter pylori and A2 and B subunit genes of Vibrio cholerae toxin, whose nucleotide sequence is represented as following(SEQ ID NO:1), or its functional equivalents:

| | |
|---|---|
| ATGAAAAAAA CAGCTATCGC GATTGCAGTG GCACTGGCTG GTTTCGCTTC | 50 |
| CGTGGCTCAA GCTGCATGGA AAAAATGCCT TTTAGGCGCG AGCGTGGTCG | 100 |
| CTTTATTAGT GGGATGCAGC CCGCATATTA TTGAAACCAA TGAAGTCGCT | 150 |
| TTGAAATTGA ATTACCATCC AGCTAGCGAG AAAGTTCAAG CGTTAGATGA | 200 |
| AAAGATTTTG CTTTTAAGGC CAGCTTTCCA ATATAGCGAT AATATCGCTA | 250 |
| AAGAGTATGA AAACAAATTC AAGAATCAAA CCGCGCTCAA GGTTGAACAG | 300 |
| ATTTTGCAAA ATCAAGGCTA TAAGGTTATT AGCGTAGATA GCAGCGATAA | 350 |
| AGACGATTTT TCTTTTGCAC AAAAAAAAGA AGGGTATTTG GCGGTTGCTA | 400 |
| TGAATGGCGA AATTGTTTTA CGCCCCGATC CTAAAAGGAC CATACAGAAA | 450 |
| AAATCAGAAC CCGGGTTATT ATTCTCCACC GGTTTGGACA AAATGGAAGG | 500 |
| GGTTTTAATC CCGGCTGGGT TTATTAAGGT TACCATACTA GAGCCTATGA | 550 |
| GTGGGGAATC TTTGGATTCT TTTACGATGG ATTTGAGCGA GTTGGACATT | 600 |
| CAAGAAAAAT TCTTAAAAAC CACCCATTCA AGCCATAGCG GGGGGTTAGT | 650 |
| TAGCACTATG GTTAAGGGAA CGGATAATTC TAATGACGCG ATCAAGAGCC | 700 |
| CTTTGAATAA GATTTTTGCA AATATCATGC AAGAAATAGA CAAAAAACTC | 750 |
| ACTCAAAAGA ATTTAGAATC TTATCAAAAA GACGCCAAAG AATTAAAAGG | 800 |
| CAAAAGAAAC CGAGAATTCG AGCCGTGGAT TCATCATGCA CCGCCGGGTT | 850 |
| GTGGGAATGC TCCAAGATCA TCGATCAGTA ATACTTGCGA TGAAAAAACC | 900 |
| CAAAGTCTAG GTGTAAAATT CCTTGACGAA TACCAATCTA AAGTTAAAAG | 950 |
| ACAAATATTT TCAGGCTATC AATCTGATAT TGATACACAT AATAGAATTA | 1000 |
| AGGATGAATT ATGATTAAAT TAAAATTTGG TGTTTTTTTT ACAGTTTTAC | 1050 |
| TATCTTCAGC ATATGCACAT GGAACACCTC AAAATATTAC TGATTTGTGT | 1100 |
| GCAGAATCAC ACAACACACA AATATATACG CTAAATGATA AGATATTTTC | 1150 |
| GTATACAGAA TCTCTAGCTG GAAAAAGAGA GATGGCTATC ATTACTTTTA | 1200 |
| AGAATGGTGC AATTTTTCAA GTAGAAGTAC CAAGTAGTCA ACATATAGAT | 1250 |
| TCACAAAAAA AAGCGATTGA AAGGATGAAG GATACCCTGA GGATTGCATA | 1300 |
| TCTTACTGAA GCTAAAGTCG AAAAGTTATG TGTATGGAAT AATAAAACGC | 1350 |
| CTCATGCGAT TGCCGCAATT AGTATGGCAA ATTAAGATAT AAAAAGCCCA | 1400 |
| CCTCAGTGGG CTTTTTTGTG GTTCGATGAT GAGAAGCAAC CGTTTTGCCC | 1450 |
| AAACATGTAT TACTGCAAGT ATGATGTTTT TATTCCACAT CCTTAGTGCG | 1500 |
| TATTATGTGC TGCAGT | 1516 |

2. A protein having the amino acid sequence represented as following(SEQ ID NO:2) which is deducable from the recombinant DNA of claim 1 or its functionally equivalent amino acid sequence:

| | |
|---|---|
| Met Lys Lys Thr Ala Ile Ala Ile Ala Val Ala Leu Ala | 13 |
| Gly Phe Ala Ser Val Ala Gln Ala Ala Trp Lys Lys Cys | 26 |
| Leu Leu Gly Ala Ser Val Val Ala Lys Lys Val Gly Cys | 39 |
| Ser Pro His Ile Ile Glu Thr Asn Glu Val Ala Leu Lys | 52 |
| Leu Asn Tyr His Pro Ala Ser Glu Lys Val Gln Ala Leu | 65 |
| Asp Glu Lys Ile Leu Leu Leu Arg Pro Ala Phe Gln Tyr | 78 |
| Ser Asp Asn Ile Ala Lys Glu Tyr Glu Asn Lys Phe Lys | 91 |
| Asn Gln Thr Ala Leu Lys Val Glu Gln Ile Leu Gln Asn | 104 |
| Gln Gly Tyr Lys Val Ile Ser Val Asp Ser Ser Asp Lys | 117 |
| Asp Asp Phe Ser Phe Ala Gln Lys Lys Glu Gly Tyr Leu | 130 |
| Ala Val Ala Met Asn Gly Glu Ile Val Leu Arg Pro Asp | 143 |
| Pro Lys Arg Thr Ile Gln Lys Lys Ser Glu Pro Gly Leu | 156 |
| Leu Phe Ser Thr Gly Lys Asp Lys Met Glu Gly Val Leu | 169 |
| Ile Pro Ala Gly Phe Ile Lys Val Thr Ile Leu Glu Pro | 182 |
| Met Ser Gly Glu Ser Leu Asp Ser Phe Thr Met Asp Leu | 195 |
| Ser Glu Leu Asp Ile Gln Glu Lys Phe Leu Lys Thr Thr | 208 |
| His Ser Ser His Ser Gly Gly Leu Val Ser Thr Met Val | 221 |
| Lys Gly Thr Asp Asn Ser Asn Asp Ala Ile Lys Ser Ala | 234 |
| Leu Asn Lys Ile Phe Ala Asn Ile Met Gln Glu Ile Asp | 247 |
| Lys Lys Leu Thr Gln Lys Asn Leu Glu Ser Thr Gly Lys | 260 |
| Asp Ala Lys Gly Leu Lys Gly Lys Arg Asn Arg Glu Phe | 273 |
| Glu Pro Trp Ile His His Ala Pro Pro Gly Cys Gly Asn | 286 |
| Ala Pro Arg Ser Ser Ile Ser Asn Thr Cys Asp Glu Lys | 299 |
| Thr Gln Ser Leu Gly Val Lys Phe Leu Asp Glu Tyr Gln | 312 |
| Ser Lys Val Lys Arg Gln Ile Phe Ser Gly Tyr Gln Ser | 325 |
| Asp Ile Asp Thr His Asn Arg Ile Lys Asp Glu Leu Met | 338 |
| Ile Lys Leu Lys Phe Gly Val Phe Phe Thr Val Leu Leu | 351 |
| Ser Ser Ala Tyr Ala His Gly Thr Pro Gln Asn Ile Thr | 364 |
| Asp Leu Cys Ala Glu Ser His Asn Thr Gln Ile Tyr Thr | 377 |
| Leu Asn Asp Lys Ile Phe Ser Tyr Thr Glu Ser Leu Ala | 390 |
| Gly Lys Arg Glu Met Ala Ile Ile Thr Phe Lys Asn Gly | 403 |
| Ala Ile Phe Glu Val Gly Val Pro Ser Ser Gln His Ile | 416 |
| Asp Ser Gln Lys Lys Ala Ile Glu Arg Met Lys Asp Thr | 429 |
| Leu Arg Ile Ala Tyr Leu Thr Glu Ala Lys Val Glu Lys | 442 |
| Leu Cys Val Trp Asn Asn Lys Thr Pro His Ala Ile Ala | 455 |
| Ala Ile Ser Met Ala Asn | 461 |

3. A pHA022 expression vector which can express the protein having the amino acid sequence of claim 2.

4. Escherichia coli DW/HP-222(KCCM-10078) which is transformed with the pHA022 expression vector of claim 3.

5. A process for preparing a chimeric protein of adhesin of H. pylori and A2 and B subunits of a Vibrio cholerae toxin, which comprises the steps of culturing a host microorganism transformed with the recombinant DNA of claim 1, and obtaining the chimeric protein.

6. The process for preparing the chimeric protein of claim 5, wherein the transformed host microorganism is Escherichia coli DW/HP-222(KCCM-10078).

7. A chimeric protein of adhesin of H. pylori and A2 and B subunits of a Vibrio cholerae toxin prepared by the process comprising a step of culturing a host microorganism transformed with the recombinant DNA of claim 1, and obtaining the chimeric protein.
